SEQUENCE LISTING (67 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 19 bp, Homo sapiens
cactgtgcgcagcttccac

SEQ ID NO: 4 - DNA, 19 bp, Homo sapiens
cagatgcaagatgctttag

SEQ ID NO: 5 - DNA, 19 bp, Homo sapiens
gaactatcaggacatggtt

SEQ ID NO: 6 - DNA, 19 bp, Homo sapiens
tgacgagaatgaaaaggtt

SEQ ID NO: 7 - DNA, 19 bp, Homo sapiens
gctgtaccttgacgagaat

SEQ ID NO: 8 - DNA, 19 bp, Homo sapiens
cgacagaactcagtgctat

SEQ ID NO: 9 - DNA, 19 bp, Homo sapiens
ggtcaactctgttaactct

SEQ ID NO: 10 - DNA, 19 bp, Homo sapiens
cactaatcatgccattgtt

SEQ ID NO: 11 - DNA, 19 bp, Homo sapiens
ggggtggaatgactggatt

SEQ ID NO: 12 - DNA, 19 bp, Homo sapiens
ccacaaaagagaaaaacgt

SEQ ID NO: 13 - DNA, 19 bp, Homo sapiens
gcatcctctccacaaaaga

SEQ ID NO: 14 - DNA, 19 bp, Homo sapiens
cttccaccatgaagaatct

SEQ ID NO: 15 - DNA, 19 bp, Homo sapiens
gcagcttccaccatgaaga

SEQ ID NO: 16 - DNA, 19 bp, Homo sapiens
ctacatgctagacctgtat

SEQ ID NO: 17 - DNA, 19 bp, Homo sapiens
gcatgttcggcctgaaaca

SEQ ID NO: 18 - DNA, 19 bp, Homo sapiens
gtaccttgacgagaatgaa

SEQ ID NO: 19 - DNA, 19 bp, Homo sapiens
cgatgctgtaccttgacga

SEQ ID NO: 20 - DNA, 19 bp, Homo sapiens
ctatctcgatgctgtacct

SEQ ID NO: 21 - DNA, 19 bp, Homo sapiens
tcagtgctatctcgatgct

SEQ ID NO: 22 - DNA, 19 bp, Homo sapiens
gaactcagtgctatctcga

SEQ ID NO: 23 - DNA, 19 bp, Homo sapiens
agaactcagtgctatctcg

SEQ ID NO: 24 - DNA, 19 bp, Homo sapiens
ctctaagattcctaaggca

SEQ ID NO: 25 - DNA, 19 bp, Homo sapiens
gtggaagctgcgcacagtg

SEQ ID NO: 26 - DNA, 19 bp, Homo sapiens
ctaaagcatcttgcatctg

SEQ ID NO: 27 - DNA, 19 bp, Homo sapiens
aaccatgtcctgatagttc

SEQ ID NO: 28 - DNA, 19 bp, Homo sapiens
aaccttttcattctcgtca

SEQ ID NO: 29 - DNA, 19 bp, Homo sapiens
attctcgtcaaggtacagc

SEQ ID NO: 30 - DNA, 19 bp, Homo sapiens
atagcactgagttctgtcg

SEQ ID NO: 31 - DNA, 19 bp, Homo sapiens
agagttaacagagttgacc

SEQ ID NO: 32 - DNA, 19 bp, Homo sapiens
aacaatggcatgattagtg

SEQ ID NO: 33 - DNA, 19 bp, Homo sapiens
aatccagtcattccacccc

SEQ ID NO: 34 - DNA, 19 bp, Homo sapiens
acgtttttctcttttgtgg

SEQ ID NO: 35 - DNA, 19 bp, Homo sapiens
tcttttgtggagaggatgc

SEQ ID NO: 36 - DNA, 19 bp, Homo sapiens
agattcttcatggtggaag

SEQ ID NO: 37 - DNA, 19 bp, Homo sapiens
tcttcatggtggaagctgc

SEQ ID NO: 38 - DNA, 19 bp, Homo sapiens
atacaggtctagcatgtag

SEQ ID NO: 39 - DNA, 19 bp, Homo sapiens
tgtttcaggccgaacatgc

SEQ ID NO: 40 - DNA, 19 bp, Homo sapiens
ttcattctcgtcaaggtac

SEQ ID NO: 41 - DNA, 19 bp, Homo sapiens
tcgtcaaggtacagcatcg

SEQ ID NO: 42 - DNA, 19 bp, Homo sapiens
aggtacagcatcgagatag

SEQ ID NO: 43 - DNA, 19 bp, Homo sapiens
agcatcgagatagcactga

SEQ ID NO: 44 - DNA, 19 bp, Homo sapiens
tcgagatagcactgagttc

SEQ ID NO: 45 - DNA, 19 bp, Homo sapiens
cgagatagcactgagttct

SEQ ID NO: 46 - DNA, 95 bp, Homo sapiens
gttgttttcccactcgattctggtagttcttccaaagattcttcatggtggaagctgcgcacagtgttggctaggctggctgccctctccaaccc

SEQ ID NO: 47 - DNA, 64 bp, Homo sapiens
agcttttccaaaaagaactatcaggacatggtttctcttgaaaaccatgtcctgatagttcggg

SEQ ID NO: 48 - DNA, 64 bp, Homo sapiens
agcttttccaaaaatgacgagaatgaaaaggtttctcttgaaaaccttttcattctcgtcaggg

SEQ ID NO: 49 - DNA, 64 bp, Homo sapiens
agcttttccaaaaagctgtaccttgacgagaattctcttgaaattctcgtcaaggtacagcggg

SEQ ID NO: 50 - DNA, 64 bp, Homo sapiens
agcttttccaaaaacgacagaactcagtgctattctcttgaaatagcactgagttctgtcgggg

SEQ ID NO: 51 - DNA, 64 bp, Homo sapiens
agcttttccaaaaaggtcaactctgttaactcttctcttgaaagagttaacagagttgaccggg

SEQ ID NO: 52 - DNA, 64 bp, Homo sapiens
agcttttccaaaaacactaatcatgccattgtttctcttgaaaacaatggcatgattagtgggg

SEQ ID NO: 53 - DNA, 64 bp, Homo sapiens
agcttttccaaaaaggggtggaatgactggatttctcttgaaaatccagtcattccaccccggg

SEQ ID NO: 54 - DNA, 64 bp, Homo sapiens
agcttttccaaaaaccacaaaagagaaaaacgttctcttgaaacgtttttctcttttgtggggg

SEQ ID NO: 55 - DNA, 64 bp, Homo sapiens
agcttttccaaaaagcatcctctccacaaaagatctcttgaatcttttgtggagaggatgcggg

SEQ ID NO: 56 - DNA, 64 bp, Homo sapiens
agcttttccaaaaacttccaccatgaagaatcttctcttgaaagattcttcatggtggaagggg

SEQ ID NO: 57 - DNA, 64 bp, Homo sapiens
agcttttccaaaaagcagcttccaccatgaagatctcttgaatcttcatggtggaagctgcggg

SEQ ID NO: 58 - DNA, 64 bp, Homo sapiens
agcttttccaaaaactacatgctagacctgtattctcttgaaatacaggtctagcatgtagggg

SEQ ID NO: 59 - DNA, 64 bp, Homo sapiens
agcttttccaaaaagcatgttcggcctgaaacatctcttgaatgtttcaggccgaacatgcggg

SEQ ID NO: 60 - DNA, 64 bp, Homo sapiens
agcttttccaaaaagtaccttgacgagaatgaatctcttgaattcattctcgtcaaggtacggg

SEQ ID NO: 61 - DNA, 64 bp, Homo sapiens
agcttttccaaaaacgatgctgtaccttgacgatctcttgaatcgtcaaggtacagcatcgggg

SEQ ID NO: 62 - DNA, 64 bp, Homo sapiens
agcttttccaaaaactatctcgatgctgtaccttctcttgaaaggtacagcatcgagatagggg

SEQ ID NO: 63 - DNA, 64 bp, Homo sapiens
agcttttccaaaaatcagtgctatctcgatgcttctcttgaaagcatcgagatagcactgaggg

SEQ ID NO: 64 - DNA, 64 bp, Homo sapiens
agcttttccaaaaagaactcagtgctatctcgatctcttgaatcgagatagcactgagttcggg

SEQ ID NO: 65 - DNA, 64 bp, Homo sapiens
agcttttccaaaaaagaactcagtgctatctcgtctcttgaacgagatagcactgagttctggg

SEQ ID NO: 66 - DNA, 64 bp, Homo sapiens
agcttttccaaaaactctaagattcctaaggcatctcttgaatgccttaggaatcttagagggg

SEQ ID NO: 67 - DNA, 19 bp, Homo sapiens
tgccttaggaatcttagag

(19) United States
(12) Patent Application Publication
(10) Pub. No.: US 2008/0249038 A1
(43) Pub. Date: Oct. 9, 2008

(54) Bone Morphogenetic Protein (Bmp) 2A and Uses Thereof

(75) Inventors: Elena Feinstein, Rehovot (IL); Igor Mett, Rehovot (IL); Svetlana Gorodin, Rishon-le-Zion (IL); Michael Shtutman, Rehovot (IL)

Correspondence Address: COOPER & DUNHAM, LLP, 1185 AVENUE OF THE AMERICAS, NEW YORK, NY, 10036, US

(73) Assignee: Quark Biotech, Inc., Fremont, CA (US)

(21) Appl. No.: 10/575,121

(22) PCT Filed: Oct. 6, 2004
(86) PCT No.: PCT/IL04/00924

§ 371 Date: Apr. 7, 2006

Related U.S. Application Data
(60) Provisional application No. 60/509,279, filed on Oct. 7, 2003.

(51) Int. Cl. A61K 31/7088; C07H 21/04; C12N 15/00

(52) U.S. Cl. 514/44; 536/23.1; 435/320.1

(57) ABSTRACT

The present invention provides compositions and methods for alleviation or reduction of the symptoms and signs associated with damaged neuronal tissues whether resulting from tissue trauma, or from chronic or acute degenerative changes. In particular, some embodiments of the present invention provide one or more pharmaceutical compositions comprising as an active ingredient a BMP2A inhibitor further comprising a pharmaceutically acceptable diluent or carrier. An additional embodiment provides a method for reducing damage to the central nervous system in a patient who has suffered an injury to the central nervous system, comprising administering to the patient a pharmaceutical composition in a dosage sufficient to reduce the damage. Yet another embodiment provides of the use of a BMP2A inhibitor for the preparation of a medicament for promoting or enhancing recovery in a patient who has suffered an injury to the central nervous system. Preferable inhibitors according to some embodiments of the invention are siRNA molecules and neutralizing antibodies. An additional embodiment provides a method for identifying a chemical compound that modulates apoptosis. Further, a process for diagnosing a neurodegenerative disease or an ischemic event in a subject is provided. The preferred methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### FIELD OF THE INVENTION

The present invention relates to the field of treatment of ischemic and neurotoxic events, particularly of the central nervous system.

### BACKGROUND OF THE INVENTION

### Ischemia of the Brain

Brain injury such as trauma and stroke are among the leading causes of mortality and disability in the western world.

Traumatic brain injury (TBI) is one of the most serious reasons for hospital admission and disability in modern society. Clinical experience suggests that TBI may be classified into primary damage occurring immediately after injury, and secondary damage, which occurs during several days post injury. Current therapy of TBI is either surgical or else mainly symptomatic.

Cerebrovascular diseases occur predominately in the middle and late years of life. They cause approximately 200,000 deaths in the United States each year as well as considerable neurologic disability. The incidence of stroke increases with age and affects many elderly people, a rapidly growing segment of the population. These diseases cause either ischemia-infarction or intracranial hemorrhage.

Stroke is an acute neurologic injury occurring as a result of interrupted blood supply, resulting in an insult to the brain. Most cerebrovascular diseases present as the abrupt onset of focal neurologic deficit. The deficit may remain fixed, or it may improve or progressively worsen, leading usually to irreversible neuronal damage at the core of the ischemic focus, whereas neuronal dysfunction in the penumbra may be treatable and/or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

Neuroprotective drugs are being developed in an effort to rescue neurons in the penumbra from dying, though as yet none has been proven efficacious.

Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

### BMP2A

Bone morphogenic protein binds the BMP I/II heterodimer receptor and may play a part in activating SMAD and NF-kappa b pathways. The activation of NF-kappa B via TGF-beta activated kinase (TAK1) provides a proapoptotic signal (Kimura N, Matsuo R, Shibuya H, Nakashima K, Taga T: BMP2-induced apoptosis is mediated by activation of the TAK1-p38 kinase pathway that is negatively regulated by Smad6. J Biol Chem. 2000 Jun. 9;275(23):17647-52). There is also evidence that BMP induces apoptosis via the PKC dependent pathway (Hay E, Lemonnier J, Fromigue O, Marie P J: Bone morphogenetic protein-2 promotes osteoblast apoptosis through a Smad-independent, protein kinase C-dependent signaling pathway. J Biol Chem. 2001 Aug. 3;276(31):29028-36.). BMP-2 plays an important role in different stages of brain development (Stull N D, Jung J W, Iacovitti L: Induction of a dopaminergic phenotype in cultured striatal neurons by bone morphogenetic proteins. Brain Res Dev. 2001 Sep. 23;130(1):91-8; Gratacos E, Checa N, Alberch J: Bone morphogenetic protein-2, but not bone morphogenetic protein-7, promotes dendritic growth and calbindin phenotype in cultured rat striatal neurons, Neuroscience. 2001;104(3):783-90; Nakashima K, Takizawa T, Ochiai W, Yanagisawa M, Hisatsune T, Nakafuku M, Miyazono K, Kishimoto T, Kageyama R, Taga T: BMP2-mediated alteration in the developmental pathway of fetal mouse brain cells from neurogenesis to astrocytogenesis. Proc Natl Acad Sci USA. 2001 May 8;98(10):5868-73.). In primary human calvaria osteoblasts & in immortalized human neonatal calvaria osteoblasts, BMP2A promotes apoptosis. Studies of the BMP2 apoptosis related mechanisms of action have shown that BMP2A increases the Bax/Bcl-2 ratio. Moreover, BMP2A increases the release of mitochondrial cytochrom C to the cytosol. In addition and consistently with these findings, BMP2A increases caspase-9 & caspase-3, -6 & -7 activity. The proapoptotic effect of BMP2A is PKC dependant.

WO 2002022871 discloses novel human bone morphogenetic protein 2, useful for the treatment, diagnosis or prediction of the clinical course of osteoporosis;

U.S. Pat. No. 6,245,889 discloses new purified bone morphogenetic protein-4, useful for treating bone (e.g. osteoporosis) or cartilage defects, for inducing bone and/or cartilage formation, as well as in wound healing and related tissue repair;

U.S. Pat. No. 6,150,328 discloses methods for Inducing bone and/or cartilage formation for wound healing and tissue repair, which involve administering a purified bone morphogenic protein produced by culturing a cell transformed with DNA encoding BMP;

WO 2000017360 concerns new mutant cystine knot growth factor proteins comprising one or more mutant subunits, useful for treating or preventing diseases e.g. hypothyroidism and thyroid cancer;

U.S. Pat. No. 5,618,924 discloses proteins BMP-2A and BMP-2B—for treating bone and cartilage defects, etc.;

U.S. Pat. No. 5,631,142 provides for the production of human bone morphogenic protein 2A or 2B in cell culture—useful for inducing bone or cartilage production, in wound healing and tissue repair;

WO 9309229 concerns recombinant hetero-dimeric BMP proteins, useful in treating bone defects, healing bone injury and in wound healing;

U.S. Pat. No. 5,166,058 provides for DNA encoding osteo-inductive proteins—used for producing BMP-2A and BMP-2B for inducing bone or cartilage formation and wound healing;

U.S. Pat. No. 5,013,649 discloses new DNA sequences encoding osteo-inductive protein—useful for stimulating bone and cartilage re formation e.g. for wound healing and tissue repair;

WO 9403600 concerns a morphogenic protein soluble complex—for regeneration of tissue in mammals and diagnosing tissue disorders;

WO 2001053486 provides for thirty five nucleic acids encoding PRO polypeptides, useful for treating benign or malignant tumours, leukaemias and lymphoid malignancies, inflammatory, angiogenic and immunologic disorders;

U.S. Pat. No. 5,863,758 discloses nucleic acids encoding mammalian osteogenic proteins in prepro form—able able to induce cartilage and bone formation when implanted in matrix, useful for repairing bone defects;

U.S. Pat. No. 5,958,441 provides for an Implant for mammals permitting the influx, proliferation and differentiation of migratory progenitor cells, useful for inducing endochondral bone formation in mammals;

U.S. Pat. No. 5,714,589 deals with the extraction of osteogenic protein from mixture—using antibodies specific for novel polypeptide chains useful as subunit(s) of dimeric osteogenic protein(s);

U.S. Pat. No. 5,468,845 concerns antibodies with osteogenic protein binding specificity—used in purification of osteogenic proteins, and as antigenic proteins;

U.S. Pat. No. 5,266,683 discloses new pure mammalian osteogenic proteins which induce cartilage and endochondral bone formation when in association with a matrix;

WO 8800205 discloses bone morphogenic proteins—obtd. using recombinant DNA and used for inducing cartilage and bone formation; and

U.S. Pat. No. 5,354,557 concerns an implantable device for inducing osteogenesis which comprises porous matrix containing non-glycosylated dimeric, disulphide linked osteogenic protein.

None of the above publications disclose a role for BMP2 in connection with neurotoxic events or the diagnosis or treatment of neurodegenerative diseases such as, inter alia, stroke, and certainly no role for BMP2A in connection with these diseases.

### SUMMARY OF THE INVENTION

The present invention provides compositions and methods for alleviation or reduction of the symptoms and signs associated with damaged neuronal tissues whether resulting from tissue trauma, or from chronic or acute degenerative changes.

In particular, some embodiments of the present invention provide one or more pharmaceutical compositions comprising as an active ingredient a BMP2A inhibitor further comprising a pharmaceutically acceptable diluent or carrier.

An additional embodiment provides a method for reducing damage to the central nervous system in a patient who has suffered an injury to the central nervous system, comprising administering to the patient a pharmaceutical composition in a dosage sufficient to reduce the damage. Yet another embodiment provides for the use of a BMP2A inhibitor for the preparation of a medicament for promoting or enhancing recovery in a patient who has suffered an injury to the central nervous system. Preferable inhibitors according to some embodiments of the invention are siRNA molecules and neutralizing antibodies.

An additional embodiment provides a method for identifying a chemical compound that modulates apoptosis.

Further, a process for diagnosing a neurodegenrative disease or an ischemic event in a subject is provided.

The preferred methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### DETAILED DESCRIPTION OF THE INVENTION

The present invention, in some of its embodiments, provides polynucleotides, oligonucleotides, polypeptides, small molecules, compositions and methods for alleviation or reduction of the symptoms and signs associated with damaged neuronal tissues whether resulting from tissue trauma, or from acute and chronic degenerative changes. Certain aspects of the present invention provide pharmaceutical compositions which reduce or even completely diminish tissue damage or degeneration. In additional aspects, the present invention provides methods leading to functional improvement after traumatic ischemic events. These effects are achieved by administering an agent that inhibits the biological activity of BMP2A or the expression of BMP2A.

The inventors of the present invention discovered that the expression of BMP2A is involved in apoptosis induced by hydrogen peroxide, which is caused by oxidative stress, and that anti-sense BMP2A RNA protected the cells from this apoptosis.

Without being bound by theory, applicants suggest that BMP2A inhibitor can prevent neurotoxic-stress induced apoptosis of neurons that occurs during an ischemic event, and thus contribute to preventing the damage caused by said ischemic event.

The term “apoptosis” is particularly defined as execution of a built-in cell death program resulting in chromatin fragmentation into membrane-bound particles, changes in cell cytoskeleton and membrane structure and subsequent phagocytosis of apoptotic cell by other cells. However, as used herein, it should be understood that this term should be construed more broadly as encompassing neuronal cell death, whether or not that cell death is strictly by means of the apoptotic process described above

The term “BMP2A”, as used herein, refers to the expressed polypeptide of the BMP2A gene, derived from any organism, preferably man, and homologs (including the murine homolog) and fragments thereof having similar biological activity. Polypeptides encoded by nucleic acid sequences which bind to the BMP2A gene under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. The cDNA sequence and amino acid sequence of BMP2A are set out in FIGS. 1 and 2 respectively. Particular fragments of BMP2A include amino acids 1-50, 51-100, 101-150, 151-200, 201-250, 251-300, 301-350 and 351-396 of the sequence shown in FIG. 2. Further particular fragments of BMP2A include amino acids 25-74, 75-124, 125-174, 175-224, 225-274, 275-324, 325-374 and 375-396 of the sequence shown in FIG. 2.

While the term BMP2A is mostly used herein, it is to be understood that for all exemplary embodiments, other members of the BMP protein family can effectively replace BMP2A, or act in conjunction with or in addition to BMP2A, so long as said BMP family protein possesses BMP2A biological activity as described herein.

By “biological effect of BMP2A” or “BMP2A biological activity” is meant the effect of BMP2A in apoptosis, also termed “BMP2A-induced apoptosis” herein, which may be direct or indirect, and includes, without being bound by theory, the effect of BMP2A on apoptosis induced by neurotoxic stress. The indirect effect includes, but is not limited to, BMP2A binding to or having an effect on one of several molecules, which are involved in a signal transduction cascade resulting in apoptosis.

By “BMP2A inhibitor” is meant any molecule, whether a polynucleotide, oligonucleotide, polypeptide, antibody, or small chemical compound, that prevents or reduces the biological effect of BMP2A, as recited above. BMP2A inhibitor may also be an inhibitor of the BMP2A promoter or of BMP2A transcription/translation such as an antisense RNA molecule, siRNA, dominant negative peptide, inter alia.

One aspect of the present invention provides for a pharmaceutical composition comprising as an active ingredient a BMP2A inhibitor in a therapeutically effective amount, which may be a small chemical compound; a polynucleotide, such as an antisense polynucleotide comprising consecutive nucleotides having a sequence which is an antisense sequence to the sequence set forth in FIG. 1 (SEQ ID No: 1), optionally having the sequence set forth in SEQ ID NOS: 46-66), or an oligonucleotide that functions as a small interfering RNA (siRNA), optionally having the sequence set forth in SEQ ID NOS: 3-45; a vector comprising any of these polynucleotides, preferably an expression vector, and a polypeptide, such as, inter alia, Sclerostin (Kusu et al., Sclerostin is a novel secreted osteoclast-derived bone morphogenetic protein (BMP) antagonist with unique ligand specificity, JBC, 2003), a dominant negative peptide, or an antibody, optionally a polyclonal or a monoclonal antibody, preferably a neutralizing antibody. The pharmaceutical composition may further contain a diluent or carrier.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and typically have a molecular weight of less than 2000 daltons, more preferably less than 1000 daltons or even less than 600 daltons.

The term “polynucleotide” refers to any molecule composed of DNA nucleotides, RNA nucleotides or a combination of both types, i.e. that comprises two or more of the bases guanidine, citosine, timidine, adenine, uracil or inosine, inter alia. A polynucleotide may include natural nucleotides, chemically modified nucleotides and synthetic nucleotides, or chemical analogs thereof. The term includes “oligonucleotides” and encompasses “nucleic acids”.

By the term “antisense” (AS) or “antisense fragment” is meant a nucleic acid fragment having inhibitory antisense activity, said activity causing a decrease in the expression of the endogenous genomic copy of the corresponding gene (in this case BMP2A). The sequence of the AS is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996: Antisense strategies in the treatment of leukemias. Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive. The AS fragment of the present invention optionally has the sequence depicted in FIG. 3 or a homologous sequence thereof. Particular AS fragments are the AS of the DNA encoding the particular fragments of BMP2A described above. For delivery of AS fragments see Example 8.

By “small interfering RNA” (siRNA) is meant an RNA molecule which decreases or silences (prevents) the expression of a gene/ mRNA of its endogenous or cellular counterpart. In the context of the present invention, a BMP2A siRNA is an siRNA which down-regulates the expression of the BMP2A gene. The term is understood to encompass “RNA interference” (RNAi), and “double-stranded RNA” (dsRNA). Further, the term is understood to include siRNA molecules which contain bases modified by a wide array of possible modifications, such as those described herein. For recent information on these terms and proposed mechanisms, see Bernstein E., Denli A M., Hannon G J: The rest is silence. RNA. 2001 Nov.;7(11):1509-21; Nishikura K.: A short primer on RNAi: RNA-directed RNA polymerase acts as a key catalyst. Cell. 2001 Nov. 16;107(4):415-8; and PCT publication WO 01/36646 (Glover et al).

General Specifications of siRNAs of the Present Invention

Generally, the siRNAs used in the present invention comprise a ribonucleic acid comprising a double stranded structure, whereby the double-stranded structure comprises a first strand and a second strand, whereby the first strand comprises a first stretch of contiguous nucleotides and whereby said first stretch is at least partially complementary to a target nucleic acid, and the second strand comprises a second stretch of contiguous nucleotides and whereby said second stretch is at least partially identical to a target nucleic acid, whereby said first strand and/or said second strand comprises a plurality of groups of modified nucleotides having a modification at the 2′-position whereby within the strand each group of modified nucleotides is flanked on one or both sides by a flanking group of nucleotides whereby the flanking nucleotides forming the flanking group of nucleotides is either an unmodified nucleotide or a nucleotide having a modification different from the modification of the modified nucleotides. Further, said first strand and/or said second strand may comprise said plurality of modified nucleotides and may comprises said plurality of groups of modified nucleotides.

The group of modified nucleotides and/or the group of flanking nucleotides may comprise a number of nucleotides whereby the number is selected from the group comprising one nucleotide to 10 nucleotides. In connection with any ranges specified herein it is to be understood that each range discloses any individual integer between the respective figures used to define the range including said two figures defining said range. In the present case the group thus comprises one nucleotide, two nucleotides, three nucleotides, four nucleotides, five nucleotides, six nucleotides, seven nucleotides, eight nucleotides, nine nucleotides and ten nucleotides.

The pattern of modified nucleotides of said first strand may be the same as the pattern of modified nucleotides of said second strand, and may align with the pattern of said second strand. Additionally, the pattern of said first strand may be shifted by one or more nucleotides relative to the pattern of the second strand.

The modifications discussed above may be selected from the group comprising amino, fluoro, methoxy, alkoxy and alkyl.

The double stranded structure of the siRNA may be blunt ended, on one or both sides. More specifically, the double stranded structure may be blunt ended on the double stranded structure's side which is defined by the S′-end of the first strand and the 3′-end of the second strand, or the double stranded structure may be blunt ended on the double stranded structure's side which is defined by at the 3′-end of the first strand and the 5′-end of the second strand.

Additionally, at least one of the two strands may have an overhang of at least one nucleotide at the 5′-end; the overhang may consist of at least one deoxyribonucleotide. At least one of the strands may also optionally have an overhang of at least one nucleotide at the 3′-end.

The length of the double-stranded structure of the siRNA is typically from about 17 to 24 and more preferably 18 or 19 bases. Further, the length of said first strand and/or the length of said second strand may independently from each other be selected from the group comprising the ranges of from about 15 to about 23 bases, 17 to 21 bases and 18 or 19 bases.

Additionally, the complementarily between said first strand and the target nucleic acid may be perfect, or the duplex formed between the first strand and the target nucleic acid may comprise at least 15 nucleotides wherein there is one mismatch or two mismatches between said first strand and the target nucleic acid forming said double-stranded structure.

In some cases both the first strand and the second strand each comprise at least one group of modified nucleotides and at least one flanking group of nucleotides, whereby each group of modified nucleotides comprises at least one nucleotide and whereby each flanking group of nucleotides comprising at least one nucleotide with each group of modified nucleotides of the first strand being aligned with a flanking group of nucleotides on the second strand, whereby the most terminal S′ nucleotide of the first strand is a nucleotide of the group of modified nucleotides, and the most terminal 3′ nucleotide of the second strand is a nucleotide of the flanking group of nucleotides. Each group of modified nucleotides may consist of a single nucleotide and/or each flanking group of nucleotides may consist of a single nucleotide.

Additionally, it is possible that on the first strand the nucleotide forming the flanking group of nucleotides is an unmodified nucleotide which is arranged in a 3′ direction relative to the nucleotide forming the group of modified nucleotides, and on the second strand the nucleotide forming the group of modified nucleotides is a modified nucleotide which is arranged in 5′ direction relative to the nucleotide forming the flanking group of nucleotides.

Further the first strand of the siRNA may comprise eight to twelve, preferably nine to eleven, groups of modified nucleotides, and the second strand may comprise seven to eleven, preferably eight to ten, groups of modified nucleotides.

The first strand and the second strand may be linked by a loop structure, which may be comprised of a non-nucleic acid polymer such as, inter alia, polyethylene glycol. Alternatively, the loop structure may be comprised of a nucleic acid.

Further, the 5′-terminus of the first strand of the siRNA may be linked to the 3′-terminus of the second strand, or the 3′-end of the first strand may be linked to the 5′-terminus of the second strand.

Particular Specifications of siRNAs of the Present Invention

In particular, the siRNAs used in the present invention are an oligoribonucleotide wherein one strand comprises consecutive nucleotides having, from 5′ to 3′, the sequence set forth in SEQ ID NO: 3-24 (which are sense strands) wherein a plurality of the bases may be modified, preferable 2-O-methyl modified, or a homolog thereof wherein in up to 2 of the nucleotides in each terminal region a base is altered.

The terminal region of the oligonucleotide refers to bases 1-4 and/or 16-19 in the 19-mer sequences.

Additionally, the siRNAs used in the present invention are oligoribonucleotides wherein one strand comprises consecutive nucleotides having, from 5′ to 3′, the sequence set forth in SEQ ID NO: 25-45 (antisense strands) or a homolog thereof wherein in up to 2 of the nucleotides in each terminal region a base is altered.

Thus, in particular aspects the oligonucleotide comprises a double-stranded structure, whereby such double-stranded structure comprises a first strand and a second strand, whereby the first strand comprises a first stretch of contiguous nucleotides and the second strand comprises a second stretch of contiguous nucleotides, whereby the first stretch is either complementary or identical to a nucleic acid sequence coding for gene BMP2A and whereby the second stretch is either identical or complementary to a nucleic acid sequence coding for BMP2A. Said first stretch comprises at least 14 nucleotides, preferably at least 18 nucleotides and even more preferably 19 nucleotides or even at least 21 nucleotides. In an embodiment the first stretch comprises from about 14 to 40 nucleotides, preferably about 18 to 30 nucleotides, more preferably from about 19 to 27 nucleotides and most preferably from about 19 to 23 nucleotides. In an embodiment the second stretch comprises from about 14 to 40 nucleotides, preferably about 18 to 30 nucleotides, more preferably from about 19 to 27 nucleotides and most preferably from about 19 to 23 nucleotides or even about 19 to 21 nucleotides. In an embodiment the first nucleotide of the first stretch corresponds to a nucleotide of the nucleic acid sequence coding for BMP2A, whereby the last nucleotide of the first stretch corresponds to a nucleotide of the nucleic acid sequence coding for BMP2A. In an embodiment the first stretch comprises a sequence of at least 14 contiguous nucleotides of an oligonucleotide, whereby such oligonucleotide is selected from the group comprising SEQ. ID. NOS 3-45, preferably from the group comprising the oligoribonucleotides of having the sequence of any of the serial numbers 1-2, 4-6, 14-16 and 18-22 in Table 1. Additionally specifications of the siRNA molecules used in the present invention may provide an oligoribonucleotide wherein the dinucleotide dTdT is covalently attached to the 3′ terminus, and/or in at least one nucleotide a sugar residue is modified, possibly with a modification comprising a 2′-O-methyl modification. Further, the 2′ OH group may be replaced by a group or moiety selected from the group comprising —H—OCH₃, —OCH₂CH₃, —OCH₂CH₂ CH₃, —NH2, and —F.

Additionally, the siRNAs used in the present invention may be an oligoribonucleotide wherein in alternating nucleotides modified sugars are located in both strands. Particularly, the oligoribonucleotide may comprise one of the sense strands wherein the sugar is unmodified in the terminal 5′and 3′ nucleotides, or one of the antisense strands wherein the sugar is modified in the terminal 5′and 3′ nucleotides.

Additionally, further nucleic acids to be used in the present invention comprise at least 14 contiguous nucleotides of any one of the SEQ. ID. NO. 3 to 45, and more preferably 14 contiguous nucleotide base pairs at any end of the double-stranded structure comprised of the first stretch and second stretch as described above. It will be understood by one skilled in the art that given the potential length of the nucleic acid according to the present invention and particularly of the individual stretches forming such nucleic acid according to the present invention, some shifts relative to the coding sequence of the BMP2A gene as detailed in SEQ ID NO: 1 to each side is possible, whereby such shifts can be up to 1, 2, 3, 4, 5 and 6 nucleotides in both directions, and whereby the thus generated double-stranded nucleic acid molecules shall also be within the present invention. Information on preparation of siRNAs is given in Example 3.

Thus, in connection with the siRNAs of the present invention, several embodiments and methods are provided. One embodiment provides for a double stranded oligonucleotide, preferably an oligoribonucleotide, wherein one strand comprises consecutive nucleotides having, from 5′ to 3′, the sequence set forth in SEQ ID NOS: 3-24 or a homolog thereof wherein in up to 2 of the nucleotides in each terminal region a base is altered. Additionally, a double stranded oligonucleotide, preferably an oligoribonucleotide, wherein one strand comprises consecutive nucleotides having, from 5′ to 3′, the sequence set forth in SEQ ID NOS: 25-45 or a homolog thereof wherein in up to 2 of the nucleotides in each terminal region a base is altered is also provided, as is a vector, preferably an expression vector, comprising any of these oligonucleotides or oligoribonucleotides.

Said double stranded oligonuclotides and oligoribonucleotides may be used in the preparation of a medicament for the treatment of a variety of conditions disclosed herein and, in particular, for the treatment of neurodegenerative diseases. Pharmaceutical compositions comprising these oligonucleotides or oligoribonucleotides are also a part of the present invention.

Further provided is a method of treating a neurodegenerative disease such as a stroke in a subject which comprises administering to the subject a therapeutically effective amount of a pharmaceutical composition comprising a BMP2A inhibitor so as to thereby treat the subject. The pharmaceutical compositions may comprise an oligoribonucleotide which is a BMP2A siRNA, such as any of the double stranded oligoribonucleotides described above, or an oligoribonucleotide which down regulates the expression of gene BMP2A by at least 50% as compared to a control. In particular, said siRNA may have a sequence set forth in Table 1, ID numbers 1-2, 4-6, 14-16 and 18-22. The pharmaceutical composition may further comprise a pharmaceutically acceptable carrier.

The term “expression vector” refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

By “polypeptide” is meant a molecule composed of amino acids and the term includes peptides, polypeptides, proteins and peptidomimetics.

A peptidomimetic is a compound containing non-peptidic structural elements that is capable of mimicking the biological action(s) of a natural parent peptide. Some of the classical peptide characteristics such as enzymatically scissille peptidic bonds are normally not present in a peptidomimetic.

The term “amino acid” refers to a molecule which consists of any one of the 20 naturally occurring amino acids, amino acids which have been chemically modified (see below), or synthetic amino acids.

The term “dominant negative peptide” refers to a polypeptide encoded by a cDNA fragment that encodes for a part of a protein which can interact with the full protein and inhibit its activity or which can interact with other proteins and inhibit their activity in response to the full protein.

The term “antibody” refers to IgG, IgM, IgD, IgA, and IgE antibody, inter alia. The definition includes polyclonal antibodies or monoclonal antibodies. This term refers to whole antibodies or fragments of the antibodies comprising the antigen-binding domain of the anti-GPCRV product antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination.

The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

In one embodiment of the invention, any one of the pharmaceutical compositions disclosed herein is used for alleviation or reduction of the symptoms and signs associated with damaged neuronal tissues whether resulting from tissue trauma, or from chronic degenerative changes. This embodiment concerns a method of treating a neurodegenerative disease or reducing damage to the central nervous system or promoting recovery in a patient who has suffered an injury to the central nervous system, comprising administering to the patient any one of the pharmaceutical compositions recited above, in a dosage and over a period of time sufficient to reduce the damage or promote recovery so as to thereby treat the patient. This embodiment further provides a method or process for treating a patient who has suffered an injury to the central nervous system, optionally as a result of any of the conditions or injuries described herein, comprising administering to the patient a pharmaceutical composition comprising a therapeutically effective amount of a BMP2A inhibitor, as exemplified herein, in a dosage and over a period of time sufficient to inhibit BMP2A so as to thereby treat the patient.

It is known in the art, that in certain neurological diseases (for example, brain ischemia or stroke), the blood brain barrier (BBB) is relatively open compared to that of a normal subject, thus enabling penetration of molecules to the brain, even large molecules such as macromolecules, including antibodies, which would subsequently allow interaction of said molecules with BMP2A. Further information on delivery into the brain is provided in Example 7 below.

In one aspect of this invention, the injury to the central nervous system which said pharmaceutical composition is aimed at reducing, or from which said pharmaceutical composition is attempting to promote recovery, is an ischemic episode, which may be, but is not limited to, a global or focal cerebral episode; said injury may be a stroke event or a traumatic brain injury, as discussed herein.

In another aspect of this invention, an additional pharmaceutically effective compound is administered in conjunction with the aforementioned pharmaceutical composition.

By “in conjunction with” is meant that the additional pharmaceutically effective compound is administered prior to, at the same time as, or subsequent to administration of inhibitor.

In an additional embodiment of the present invention, any one of the above pharmaceutical compositions is used for causing regeneration of neurons in a subject in need thereof. This embodiment of the present invention concerns a method for causing regeneration of neurons in a patient in need thereof, comprising administering to the patient any one of the pharmaceutical compositions recited above, in a dosage and over a period of time sufficient to reduce the damage or promote recovery.

The pharmaceutical compositions of the present invention can have application in the treatment of any disease in which neuronal degeneration or damage is involved or implicated, such as, inter alia—the following conditions: hypertension, hypertensive cerebral vascular disease, rupture of aneurysm, a constriction or obstruction of a blood vessel—as occurs in the case of a thrombus or embolus, angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor; and diseases such as stroke, Parkinson's disease, Epilepsy, Depression, ALS, Alzheimer's disease, Huntington's disease and any other disease-induced dementia (such as HIV induced dementia for example). These conditions are also referred to herein as “neurodegenerative diseases”.

One embodiment of the claimed invention provides for using a therapeutically effective amount of a BMP2A inhibitor in a process for the preparation of a medicament for the treatment of a patient who has suffered an injury to the central nervous system such as, inter alia, an ischemic episode, a stroke or a traumatic brain injury. The inhibitor may be a small chemical compound; a polynucleotide, such as an antisense polynucleotide comprising consecutive nucleotides having a sequence which is an antisense sequence to the sequence set forth in FIG. 1 (SEQ ID NO: 1), optionally having the sequence set forth in SEQ ID NOS: 46-66, or a polynucleotide that functions as small interfering RNA (siRNA) optionally having the sequence set forth in SEQ ID NOS: 3-45 and the double stranded structure as described herein; a vector comprising any of these polynucleotides, and a polypeptide, such as, inter alia, Scierostin (see above), a dominant negative peptide, or an antibody, optionally a polyclonal or a monoclonal antibody, preferably a neutralizing antibody.

The treatment regimen according to the invention is carried out, in terms of administration mode, timing of the administration, and dosage, so that the functional recovery of the patient from the adverse consequences of the ischemic events or central nervous system injury is improved; i.e., at least one of the patient's motor skills (e.g., posture, balance, grasp, or gait), cognitive skills, speech, and/or sensory perception (including visual ability, taste, olfaction, and proprioception) improve as a result of inhibitor administration according to the invention. Thus the inhibitor promotes or enhances recovery of the patient by improving at least one of these skills.

Administration of a pharmaceutical composition comprising a BMP2A inhibitor according to the invention can be carried out by any known route of administration, including intravenously, intra-arterially, subcutaneously, or intracerebrally. Using specialized formulations, it may also be possible to administer these orally or via inhalation. Suitable doses and treatment regimens for administering compositions to an individual in need thereof are discussed in detail below.

The invention can be used to treat the adverse consequences of central nervous system injuries that result from any of a variety of conditions. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from tumor, or other blood loss.

Where the ischemia is associated with stroke, it can be either global or focal ischemia, as defined below. It is believed that the administration of a pharmaceutical composition according to the invention is effective, even though administration occurs a significant amount of time following the injury.

By “ischemic episode” is meant any circumstance that results in a deficient supply of blood to a tissue. Cerebral ischemic episodes result from a deficiency in the blood supply to the brain. The spinal cord, which is also part of the central nervous system, is equally susceptible to ischemia resulting from diminished blood flow. An ischemic episode may be caused by hypertension, hypertensive cerebral vascular disease, rupture of aneurysm, a constriction or obstruction of a blood vessel—as occurs in the case of a thrombus or embolus, angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor, or other blood loss. It is expected that the invention will also be useful for treating injuries to the central nervous system that are caused by mechanical forces, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracarnial hematoma or edema). Similarly, traumatic constriction or compression can arise from the presence of a mass of abnormal tissue, such as a metastatic or primary tumor.

By “focal ischemia” as used herein in reference to the central nervous system, is meant the condition that results from the blockage of a single artery that supply blood to the brain or spinal cord, resulting in the death of all cellular elements (pan-necrosis) in the territory supplied by that artery.

By “global ischemia” as used herein in reference to the central nervous system, is meant the condition that results from general diminution of blood flow to the entire brain, forebrain, or spinal cord, which causes the death of neurons in selectively vulnerable regions throughout these tissues. The pathology in each of these cases is quite different, as are the clinical correlates.

Models of focal ischemia apply to patients with focal cerebral infarction, while models of global ischemia are analogous to cardiac arrest, and other causes of systemic hypotension.

The term “neurotoxic stress” as used herein is intended to comprehend any stress that is toxic to normal neural cells (and may cause their death or apoptosis). Such stress may be oxidative stress (hypoxia or hyperoxia) or ischemia or trauma, and/or it may involve subjecting the cells to a substance that is toxic to the cells in vivo, such as glutamate or dopamine or the Aβ protein, or any substance or treatment that causes oxidative stress. The neurotoxic substance may be endogenous or exogenous and the term neurotoxic is also intended to comprehend exposure to various known neurotoxins including organophosphorous poisoning, or any other insult of this type. In addition, neurotoxic stress may be caused by a neurodegenerative disease.

In an additional embodiment, the present invention provides for a method or process for causing regeneration of neurons in a subject in need thereof, comprising administering to the subject a pharmaceutical composition which comprises a BMP2A inhibitor as an active ingredient in a therapeutically effective amount, further comprising a diluent or carrier and optionally being any of the pharmaceutical compositions as described herein.

An additional embodiment of the present invention, referred to herein as the “screening” embodiment, concerns methods and processes for obtaining a species and/or chemical compound that modulates the biological activity of BMP2A, neurotoxic stress and/or apoptosis. One aspect of this embodiment provides a process for obtaining a species and/or chemical compound that modulates the biological activity of BMP2A, neurotoxic stress and/or apoptosis which comprises contacting a cell expressing BMP2A with a species and/or compound and determining the ability of the species and/or compound to modulate the biological activity of BMP2A, neurotoxic stress and/or apoptosis of the cell as compared to a control. The cell being examined may be modified to express BMP2A, and —without being bound by theory—apoptosis may be induced by the presence of BMP2A, or by neurotoxic stress, optionally caused by hydrogen peroxide, glutamate, dopamine, the Aβ protein or any known neurotoxin or neurotoxic treatment such as ischemia or hypoxia, or by a neurodegenerative disease such as stroke. In addition, this process may be used in order to prepare a pharmaceutical composition. The process then comprises admixing a species or compound obtained by the process recited above or a chemical analog or homolog thereof with a pharmaceutically acceptable carrier.

By cells being “modified to express” as used herein is meant that cells are modified by transfection, transduction, infection or any other known molecular biology method which will cause the cells to express the desired gene. Materials and protocols for carrying out such methods are evident to the skilled artisan.

An additional aspect of the screening embodiment provides a process of obtaining a species and/or chemical compound that modulates the biological activity of BMP2A, neurotoxic stress and/or apoptosis, which comprises:
(a) contacting cells expressing BMP2A with a plurality of species and/or chemical compounds;
(b) determining whether the biological activity of BMP2A, neurotoxic stress and/or apoptosis is modulated in the presence of the species and/or compounds, as compared to a control; and if so
(c) separately determining whether the modulation of the biological activity of BMP2A, neurotoxic stress and/or apoptosis is effected by each species and/or compound included in the plurality of species and/or compounds, so as to thereby identify the species and/or compound which modulates the biological activity of BMP2A, neurotoxic stress and/or apoptosis.

The cells in the contacting step may be modified to express the BMP2A gene, and —without being bound by theory—apoptosis may be induced spontaneously by BMP2A overexpression, or as a result of subjection of the cells to neurotoxic stress, optionally caused by hydrogen peroxide, glutamate, dopamine, the Aβ protein or any known neurotoxin or neurotoxic treatment such as ischemia or hypoxia, or by a neurodegenerative disease such as stroke. In addition, this process may be used in order to prepare a pharmaceutical composition. The process then comprises admixing a species or compound identified by the process recited above or a chemical analog or homolog thereof with a pharmaceutically acceptable carrier.

The process may additionally comprise modification of a species or compound found to modulate apoptosis by the above process to produce a compound with improved activity and admixing such compound with a pharmaceutically acceptable carrier. This additional act may be performed with a compound discovered by any of the processes which are disclosed in the screening embodiment of the present invention, so as to thereby obtain a pharmaceutical composition comprising a compound with improved activity.

Additionally, the screening embodiment of the present invention provides an in-vitro non cell-based process for obtaining a species or compound which modulates the biological activity of BMP2A, neurotoxic stress and/or apoptosis (through BMP2A) comprising:
(a) measuring the binding of BMP2A or the BMP2A gene to an interactor;
(b) contacting BMP2A or the BMP2A gene with said species or compound; and
(c) determining whether the binding of BMP2A or the BMP2A gene to said interactor is affected by said species or compound.

The in-vitro system may be subjected to apoptotic conditions, which can be induced —without being bound by theory —by causing neurotoxic stress, as a result of treatment with, inter alia, hydrogen peroxide, glutamate, dopamine, the Aβ protein or any known neurotoxin. In addition, this process may be used in order to prepare a pharmaceutical composition. The process then comprises admixing a species or compound identified by the process recited above or a chemical analog or homolog thereof with a pharmaceutically acceptable carrier.

Another aspect of the screening embodiment provided by the present invention is a kit for obtaining a species or compound which modulates the biological activity of BMP2A or the BMP2A gene, neurotoxic stress and/or apoptosis in a cell comprising:
(a) BMP2A or the BMP2A gene; and
(b) an interactor with which BMP2A or the BMP2A gene interacts;
(c) means for measuring the interaction of BMP2A or the BMP2A gene with the interactor; and
(d) means of determining whether the binding of BMP2A or the BMP2A gene to the interactor is affected by said species or compound.

Means of measuring interactions between molecules and determining the strength, affinity, avidity and other parameters of the interaction are well known in the art (see, for example, Lubert Stryer, Biochemistry, W H Freeman & Co.; 5th edition (April 2002); and “Comprehensive Medicinal Chemistry”, by various authors and editors, published by Pergamon Press).

An additional embodiment of the present invention concerns a method or process for diagnosing cells which have been subjected to neurotoxic stress and/or stroke and/or cancer, comprising assaying for RNA corresponding to a sequence of FIG. 1 or a fragment or homolog thereof, or for the expression product of a gene in which one of said sequences is a part, the finding of up-regulation of said RNA or expression product as compared to a normal control indicating the likelihood that such cells have been subjected to neurotoxic stress and/or stroke, and further the finding of down-regulation of said RNA or expression product as compared to a normal control indicating the likelihood that such cells have been subjected to a cancer or become cancerous.

The present invention further provides a method or process for diagnosing a neurodegenerative disease in a subject comprising detecting modulation of the expression level of BMP2A (for example: by detecting BMP2A in an immunoassay) or the BMP2A gene(for example: by detecting an mRNA encoding BMP2A) in the subject, as compared to a control. In one embodiment, the subject being diagnosed is suspected to have undergone a stroke.

Another embodiment of the present invention concerns a method or process for diagnosing a neurodegenerative disease in a subject comprising detecting modulation of the expression level of the BMP polypeptide in the subject as compared to a control, whereas said modulation of expression is indicative of the likelihood of neurodegenerative disease in the subject; indeed, the diagnostic methods of the present invention may be practiced on a subject suspected to have undergone a stroke.

The expression level of the polypeptide can be assessed by assaying for mRNA encoding the BMP polypeptide (such as that described in FIG. 1 or, or a fragment or homolog thereof), or by method of an immunoassay using antibodies which detect the polypeptide. Both detection of mRNA and immunoassays can be performed by methods well known in the art. Measurement of level of the BMP2 polypeptide is determined by a method selected from the group consisting of immunohistochemistry (Microscopy, Immunohistochemistry and Antigen Retrieval Methods: For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1), western blotting (Laemmeli U K: “Cleavage of structural proteins during the assembley of the head of a bacteriophage T4”, Nature 1970;227: 680-685; Egger & Bienz, “Protein (western) blotting”, Mol Biotechnol 1994; 1(3): 289-305), ELISA (Onorato et al., “Immunohistochemical and ELISA assays for biomarkers of oxidative stress in aging and disease”, Ann NY Acad Sci 1998 20; 854: 277-90), antibody microarray hybridization (Huang, “detection of multiple proteins in an antibody-based protein microarray system, Immunol Methods 2001 1; 255 (1-2): 1-13) and targeted molecular imaging (Thomas, Targeted Molecular Imaging in Oncology, Kim et al (Eds)., Springer Verlag, 2001).

Measurement of level of BMP2 polynucleotide is determined by a method selected from: RT-PCR analysis, in-situ hybridization (“Introduction to Fluorescence In Situ Hybridization: Principles and Clinical Applications”, Andreeff & Pinkel (Editors), John Wiley & Sons Inc., 1999), polynucleotide microarray and Northern blotting (Trayhurn, “Northern blotting”, Proc Nutr Soc 1996; 55(1B): 583-9; Shifman & Stein, “A reliable and sensitive method for non-radioactive Northern blot analysis of nerve growth factor MRNA from brain tissues”, Journal of Neuroscience Methods 1995; 59: 205-208). This diagnostic method may be useful, inter alia, for diagnosing patients suspected to have undergone a stroke.

By “abnormal” in the context of protein expression, is meant a difference of at least 10% in the expression levels of the polypeptide as compared to a control.

Additionally, the invention provides a method or process of treating a tumor or an auto-immune disease in a subject which comprises administering to the subject a therapeutically effective amount of a pharmaceutical composition which modulates the biological activity of BMP2A.

Further, the invention provides a method or process of treating neurodegenerative disease in a subject which comprises administering to the subject a therapeutically effective amount of a pharmaceutical composition which inhibits the biological activity of BMP2A.

The invention further provides for the use of a BMP2A modulator in the preparation of a medicament; said medicament may be used for the treatment of a neurodegenerative disease.

An additional embodiment of the present invention provides for an siRNA molecule comprising consecutive nucleotides the sequence of which is depicted Table 1 (SEQ ID NOS: 3-45), or a vector comprising said siRNA, preferably an expression vector. The vector may be particularly suited for drug delivery. In general, both the siRNA molecule and the vector borne siRNA can be used as therapeutic agents in the treatment of neurodegenerative diseases and/or neurotoxic conditions. For therapeutic delivery of siRNAs, see Example 9.

Another embodiment of the present invention provides for a substantially purified polynucleotide comprising consecutive amino acids having the sequence set forth in SEQ ID NOS: 46-66, or a sequence at least 70% homologous thereto, preferably at least 80% homologous thereto, more preferably at least 90% or 95% homologous thereto, and a vector which comprises said polynucleotide, preferably an expression vector. Said vector may be of a specific type aimed at gene therapy or targeting.

Another aspect of the present invention deals with the use of BMP2A for its capacity to enhance apoptosis. In this aspect, the invention provides for a method or process of treating a tumor or auto-immune disease in a subject by administering to the subject a therapeutically effective amount of a chemical compound, wherein the chemical compound comprises BMP2A, or the BMP2A cDNA, or a therapeutically effective amount of a chemical compound which stimulates the BMP2A cDNA or polypeptide, all separately or in combination. In this aspect, the invention further provides for the use of BMP2A or a vector comprising the BMP2A cDNA for the preparation of a medicament for promoting or enhancing recovery in a patient suffering from a tumor or auto-immune disease.

The term “conservative substitution” refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

The term “non-conservative substitution” refers to the substitution of an amino acid in one class with an amino acid from another class; for example, substitution of an Ala, a class II residue, with a class III residue such as Asp, Asn, Glu, or Gln.

By “Chemically modified” when referring to the product of the invention, is meant a product (polypeptide) wherein at least one of the amino acid residues is modified either by natural processes, such as processing or other post-translational modifications, or by chemical modification techniques which are well known in the art. Among the numerous known modifications typical, but not inclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

The term “deletion” refers to a change in sequence of either nucleotide or amino acid molecule in which one or more nucleotides or amino acid residues, respectively, are absent, as compared to the naturally occurring.

The term “insertion” or “addition” is that change in a sequence of a nucleotide or amino acid molecule resulting in the addition of one or more nucleotides or amino acid residues, respectively, as compared to the naturally occurring molecule.

The term “substitution” refers to the replacement of one or more nucleotides or amino acids by different nucleotides or amino acids, respectively. As regards amino acid sequences the substitution may be conservative or non-conservative.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726); for instance, using a window size of 20 nucleotides, a word length of 4 nucleotides, and a gap penalty of 4, computer-assisted analysis and interpretation of the sequence data, including alignment, can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

By the term “modulates” in the context of apoptosis modulation is meant either increases (promotes, enhances) or decreases (prevents, inhibits).

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### BRIEF DESCRIPTION OF THE FIGURES

FIG. 1 shows the polynucleotide sequence of the BMP2A gene (SEQ ID NO: 1);

FIG. 2 shows the corresponding amino acid sequence (SEQ ID NO: 2);

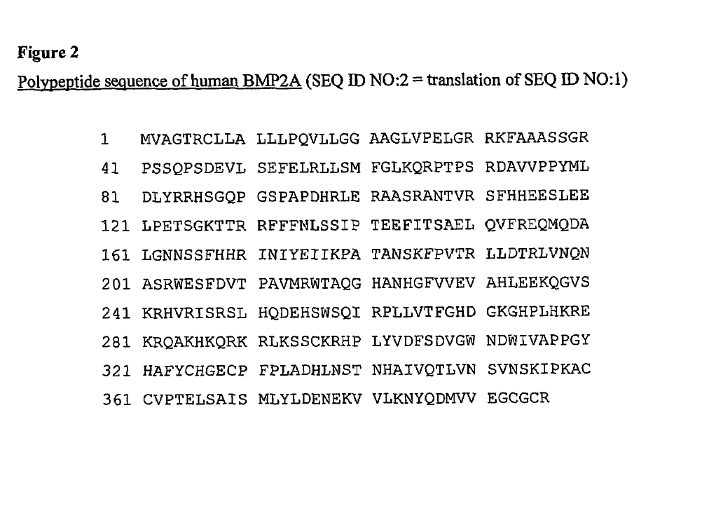

FIG. 3 is a graph illustrating the results of a loss of function validation experiment.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Example 1

### Identification of BMP2A as a Gene Important for Hypoxia-Induced Apontosis in Be2C Cells

As a first step to the novel drug discovery, key genes involved in neurotoxic stress-induced apoptosis were identified by the inventors by direct functional selection (functional profiling).

The expression libraries for functional profiling were made by cloning total cellular cDNA into retroviral expression vectors. The clones of such library may contain full-length cDNA either in sense or in the antisense orientation or cDNA fragments also transcribed either as an antisense RNA or translated as a short polypeptides that can act in a dominant negative manner. When the cDNA is expressed in the antisense orientation or as a short peptide, the result will be inhibition of the expression or activity of the matching endogenous gene. A plasmid DNA pool was prepared from the bacteria and used for the introduction of the library into mammalian retroviral packaging cells of choice. The rescued recombinant retrovirus mixture was further used for transduction of the target Be2C human neuroblastoma cells. The cDNA fragments expressed by the transduced cells can potentially inhibit or stimulate the expression of specific endogenous genes or the function of the protein expressed by such a gene. The pool of Be2C cells was subjected to G418 selection followed by induction of neuronal differentiation by treatment with retinoic acid. Differentiated Be2C cells were then treated with dopamine and/or hypoxia (oxygen deprivation) and/or glutamate at concentrations leading to 90% apoptosis. Thus, the cells were subjected to a certain selection process in which the activity of a number of genes is necessary for the cells to show a specific phenotype (eg: an apoptotic phenotype), after a specific induction (such as dopamine treatment), that can be followed experimentally (eg: cell death). When expression of a key gene is inhibited, the phenotype does not show for that cell (in an apoptotic phenotype, the cell remains viable on a background of dead cells in which the gene was not inhibited). The selection process allowed the selection of exactly these types of cells—the ones which survived by virtue of the library cDNA expression. This was followed by RT-PCR and sequencing identification of the cDNA fragments present in the expression vectors found in the cells that survived. The identity of these cDNA fragments was indicative of the identity of the inhibited gene or genes, thus identifying them as key genes required for the development of an apototic phenotype. In the direct functional profiling method, these identified fragments were used as the candidates for further analysis. One of such rescued cDNA fragments belonged to the BMP2A cDNA and was expressed in the anti-sense orientation. Thus its activity compromised or blocked the normal function of the endogenous BMP2A gene.

### Example 2

### Experimental Validation Results

Validation of the involvement of BMP2A in neruotoxic stress was conducted using BMP2A siRNA. Utilizing siRNA, one can inhibit or reduce the level of a specific desired mRNA. The siRNA of Table 1 (see below) having ID number 1 was used to successfully reduce the endogenous mRNA level of BMP2A.

Effect of siRNA on Human BMP2A Gene Expression

The effect was measured by Real-Time-PCR. The expression of Cyclophilin A serves as a reference (control) gene.

| siRNA vector | BMP2/Cyclo |
|---|---|
| siLUC | 100 |
| siBMP2-hA | 40 |

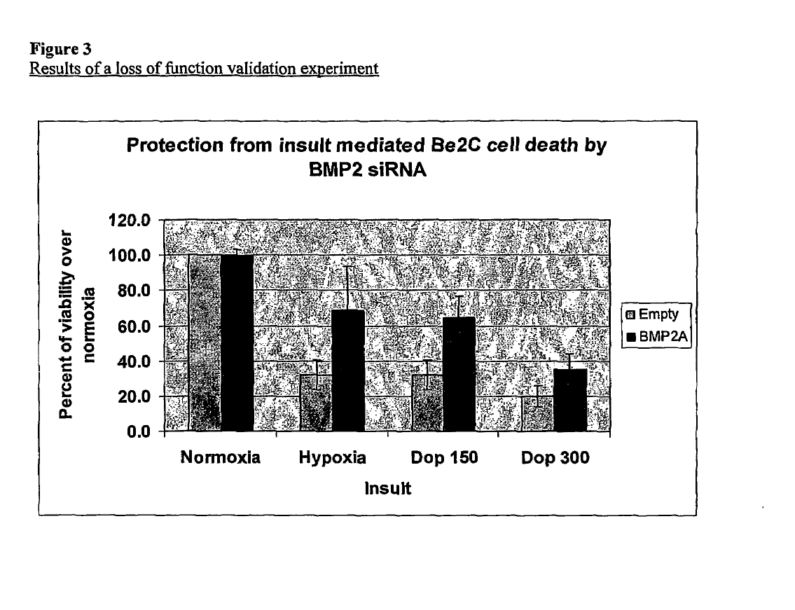

As can be seen, siBMP2-hA (a vector comprising the BMP2A siRNA depicted in FIG. 3) reduces the expression of human BMP2A by 60%.

### Loss-of-Function Validation of the Importance of BMP2A Activity for Apoptosis

Treatment of cells with dopamine leads to development of oxidative stress—a feature which also accompanies ischemia. In order to validate the involvement of BMP2A in oxidative stress induced apoptosis, BE2C cells were infected with the BMP2A siRNA (by retrovirus or lentivirus). The cells were then further subjected to treatment with dopamine or hydrogen peroxide. The stress conditions resulted in apoptosis in the control cells, but this apoptosis was significantly and greatly diminished and in the cells which contained the BMP2A siRNA (see FIG. 5). The control cells and the cells containing the BMP2A siRNA were equally viable under normal culture conditions. Viability was tested by XTT analysis.

### Example 3

### Preparation of BMP2A siRNAs

Using proprietary algorithms and the known sequence of gene BMP2A (SEQ ID NO: 1), the sequences of potential siRNAs were generated. siRNA molecules according to the above specifications were prepared essentially as described herein.

The siRNAs of the present invention can be synthesized by any of the methods which are well-known in the art for synthesis of ribonucleic (or deoxyribonucleic) oligonucleotides. For example, a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The siRNA molecules of the invention may be synthesized by procedures known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

Alternatively, the nucleic acid molecules of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siRNA molecules of the invention can also be synthesized via a tandem synthesis methodology, as described in US patent application publication No. US2004/0019001 (McSwiggen) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

For further information, see PCT publication No. WO 2004/015107 (ATUGEN).

As described above, the siRNAs of Table 1 (below) are constructed such that alternate sugars have 2′-O-methyl modification i.e. alternate nucleotides were thus modified. In these preferred embodiments, in one strand of the siRNA the modified nucleotides were numbers 1,3,5,7,9,11,13,15,17 and 19 and in the opposite strand the modified nucleotides were numbers 2,4,6,8,10,12,14,16 and 18. Thus these siRNAs are blunt-ended 19-mer RNA molecules with alternate 2-0′-methyl modifications as described above.

TABLE 1

| | | | | Corresponding gene |
|---|---|---|---|---|
| | | | | sequence gi4557368 |
| ID No | Source | Sense siRNA sequence | AntiSense siRNA sequence | (Homo sapiens) |
| 1 | Human, | CACTGTGCGCAGCTTCCAC | GTGGAAGCTGCGCACAGTG | [644-662] |
| | Rat, | | | |
| | Mouse | | | |
| 2 | Human, | CAGATGCAAGATGCTTTAG | CTAAAGCATCTTGCATCTG | [789-807] |
| | Mouse | | | |
| 3 | Human, | GAACTATCAGGACATGGTT | AACCATGTCCTGATAGTTC | [1472-1490] |
| | Rat, | | | |
| | Mouse | | | |
| 4 | Human, | TGACGAGAATGAAAAGGTT | AACCTTTTCATTCTCGTCA | [1445-1463] |
| | Rat, | | | |
| | Mouse | | | |
| 5 | Human, | GCTGTACCTTGACGAGAAT | ATTCTCGTCAAGGTACAGC | [1436-1454] |
| | Mouse | | | |
| 6 | Human, | CGACAGAACTCAGTGCTAT | ATAGCACTGAGTTCTGTCG | [1411-1429] |
| | Rat, | | | |
| | Mouse | | | |
| 7 | Human, | GGTCAACTCTGTTAACTCT | AGAGTTAACAGAGTTGACC | [1367-1385] |
| | Mouse | | | |
| 8 | Human, | CACTAATCATGCCATTGTT | AACAATGGCATGATTAGTG | [1340-1358] |
| | Rat, | | | |
| | Mouse | | | |
| 9 | Human, | GGGGTGGAATGACTGGATT | AATCCAGTCATTCCACCCC | [1247-1265] |
| | Rat, | | | |
| | Mouse | | | |
| 10 | Human, | CCACAAAAGAGAAAAACGT | ACGTTTTTCTCTTTTGTGG | [1151-1169] |
| | Rat, | | | |
| | Mouse | | | |
| 11 | Human, | GCATCCTCTCCACAAAAGA | TCTTTTGTGGAGAGGATGC | [1142-1160] |
| | Rat, | | | |
| | Mouse | | | |
| 12 | Human, | CTTCCACCATGAAGAATCT | AGATTCTTCATGGTGGAAG | [656-674] |
| | Rat, | | | |
| | Mouse | | | |
| 13 | Human, | GCAGCTTCCACCATGAAGA | TCTTCATGGTGGAAGCTGC | [652-670] |
| | Rat, | | | |
| | Mouse | | | |
| 14 | Human, | CTACATGCTAGACCTGTAT | ATACAGGTCTAGCATGTAG | [554-572] |
| | Rat, | | | |
| | Mouse | | | |
| 15 | Human, | GCATGTTCGGCCTGAAACA | TGTTTCAGGCCGAACATGC | [499-517] |
| | Rat, | | | |
| | Mouse | | | |
| 16 | Mouse | GTACCTTGACGAGAATGAA | TTCATTCTCGTCAAGGTAC | [1439-1457] |
| 17 | Human, | CGATGCTGTACCTTGACGA | TCGTCAAGGTACAGCATCG | [1432-1450] |
| | Mouse | | | |
| 18 | Human, | CTATCTCGATGCTGTACCT | AGGTACAGCATCGAGATAG | [1426-1444] |
| | Rat, | | | |
| | Mouse | | | |
| 19 | Human, | TCAGTGCTATCTCGATGCT | AGCATCGAGATAGCACTGA | [1420-1438] |
| | Rat | | | |
| 20 | Human, | GAACTCAGTGCTATCTCGA | TCGAGATAGCACTGAGTTC | [1416-1434] |
| | Rat, | | | |
| | Mouse | | | |
| 21 | Human, | AGAACTCAGTGCTATCTCG | CGAGATAGCACTGAGTTCT | [1415-1433] |
| | Rat, | | | |
| | Mouse | | | |
| 22 | Human, | CTCTAAGATTCCTAAGGCA | TGCCTTAGGAATCTTAGAG | [1382-1400] |
| | Rat, | | | |
| | Mouse | | | |

Table 1 contains 19-mer siRNAs generated by proprietary algorithms. Of these siRNAs, the preferred molecules have ID numbers 1, 2, 4, 5, 6, 14, 15, 16, 18, 19, 20, 21 and 22. In one embodiment, the molecules of ID numbers 1 and 2 are highly preferred and were used in the validation experiments detailed herein. Note that in the above Table 1, the sense strands of siRNAs 1-22 have SEQ ID NOS: 3-24 respectively, and the antisense strands of siRNAs 1-22 have SEQ ID NOS: 25-45 respectively.

### Example 4

### Preparation of Anti-BMP2A Antibodies

Antibodies which bind to BMP2A may be prepared using an intact polypeptide or fragments containing smaller polypeptides as the immunizing antigen. For example, it may be desirable to produce antibodies that specifically bind to the N- or C-terminal or any other suitable domains of BMP2A. The polypeptide used to immunize an animal can be derived from translated cDNA or chemical synthesis which can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO.91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Neutralizing antibodies can be prepared by the methods discussed above, possibly with an additional step of screening for neutralizing activity by, for example, a survival assay.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Additional information regarding all types of antibodies, including humanized antibodies, human antibodies and antibody fragments can be found in WO 01/05998, which is incorporated herein by reference in its entirety.

### Example 5

### Preparation of Polypeptides

Polypeptides may be produced via several methods, for example:
1) Synthetically:

Synthetic polypeptides can be made using a commercially available machine, using the known sequence of BMP2A.
2) Recombinant Methods:

A preferred method of making the BMP2A polypeptides is to clone a polynucleotide comprising the cDNA of the BMP2A gene into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965;23:1165-74 Appl Microbiol. 1967 Jul.;15(4):851-6; Can J Biochem. 1968 May;46(5):441-4; Biochemistry. 1968 Jul.;7(7):2574-80; Arch Biochem Biophys. 1968 Sep. 10;126(3):746-72; Biochem Biophys Res Commun. 1970 Feb. 20;38(4):825-30).).

The expression vector can include a promoter for controlling transcription of the heterologous material and can be either a constitutive or inducible promoter to allow selective transcription.

Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).
3) Purification from natural sources:

BMP2A can be purified from natural sources (such as tissues) using many methods known to one of ordinary skill in the art, such as for example: immuno-precipitation with anti-BMP2A antibody, or matrix-bound affinity chromatography with any molecule known to bind BMP2A.

Protein purification is practiced as is known in the art as described in, for example, Marshak et al., “Strategies for Protein Purification and Characterization. A laboratory course manual.” CSHL Press (1996).

### Example 6

### Preparation of Polynucleotides

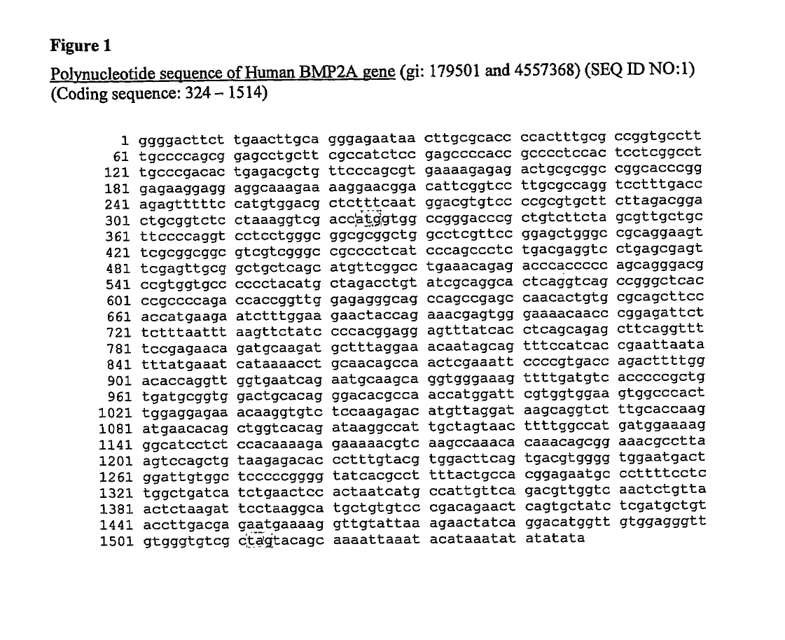

Polynucleotides of the subject invention can be constructed by using a commercially available DNA synthesizing machine and the sequence set forth FIG. 1 (SEQ ID No: 1) or fragments thereof. For example, overlapping pairs of chemically synthesized fragments can be ligated using methods well known in the art (e.g., see U.S. Pat. No. 6,121,426).

Another means of isolating a polynucleotide, e.g., the polynucleotide of FIGS. 1 or 3, is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library. The entire naturally occurring cDNA or gene sequence, including any allelic variations thereof, will all have the same utility as discussed above for the identified polynucleotide of FIGS. 1 or 3.

The polynucleotide of FIGS. 1, 3 or 4, or fragments thereof, or the oligonucleotides of Table 1 can be used inter alia as a probe for diagnostic work. They can be used to diagnose cells which have undergone stroke, neurotoxic stress or TBI, whereby said polynucleotide sequence is over-expressed and there are, thus, high levels of mRNA gene transcripts. In addition, it can be used to diagnose cells which have undergone a cancerous transformation, in which case the aforementioned polynucleotide would be under-expressed (and its level can be compared to the level in a normal subject for the purpose of diagnosis).

### Example 7

### Pharmacology and Drug Delivery

The compounds or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24-to 48 hrs or by continuous infusion during a period of 1-2 weeks or longer.

It will be appreciated that the most appropriate administration of the pharmaceutical compositions of the present invention will depend on the type of injury or disease being treated. Thus, the treatment of an acute event will necessitate systemic administration of the active composition comparatively rapidly after induction of the injury. On the other hand, treatment (diminution) of chronic degenerative damage may necessitate a sustained dosage regimen.

Delivery of BMP2A Inhibitors into the Brain

Delivery of compounds (including compounds comprising a nucleic acids or siRNA) into the brain can be accomplished by several methods such as, inter alia, neurosurgical implants, blood-brain barrier disruption, lipid mediated transport, carrier mediated influx or efflux, plasma protein-mediated transport, receptor-mediated transcytosis, absorptive-mediated transcytosis, neuropeptide transport at the blood-brain barrier, and genetically engineering “Trojan horses” for drug targeting. The above methods are performed essentially as described in “Brain Drug Targeting: the future of brain drug development”, W. M. Pardridge, Cambridge University Press, Cambridge, UK (2001).

### Example 8

### Therapeutic Delivery of Antisense Fragments

In the practice of the invention, antisense fragments may be used. The length of an antisense fragment is preferably from about 9 to about 4,000 nucleotides, more preferably from about 20 to about 2,000 nucleotides, most preferably from about 50 to about 500 nucleotides.

In order to be effective, the antisense fragments of the present invention must travel across cell membranes. In general, antisense fragments have the ability to cross cell membranes, apparently by uptake via specific receptors. As the antisense fragments are single-stranded molecules, they are to a degree hydrophobic, which enhances passive diffusion through membranes. Modifications may be introduced to an antisense fragment to improve its ability to cross membranes. For instance, the AS molecule may be linked to a group which includes partially unsaturated aliphatic hydrocarbon chain and one or more polar or charged groups such as carboxylic acid groups, ester groups, and alcohol groups. Alternatively, AS fragments may be linked to peptide structures, which are preferably membranotropic peptides. Such modified AS fragments penetrate membranes more easily, which is critical for their function and may, therefore, significantly enhance their activity. Palmityl-linked oligonucleotides have been described by Gerster et al (1998): Quantitative analysis of modified antisense oligonucleotides in biological fluids using cationic nanoparticles for solid-phase extraction. Anal Biochem. 1998 Sep. 10;262(2):177-84 Geraniol-linked oligonucleotides have been described by Shoji et al (1998): Enhancement of anti-herpetic activity of antisense phosphorothioate oligonucleotides 5′ end modified with geraniol. J Drug Target. 1998;5(4):261-73. Oligonucleotides linked to peptides, e.g., membranotropic peptides, and their preparation have been described by Soukchareun et al (1998): Use of Nalpha-Fmoc-cysteine(S-thiobutyl) derivatized oligodeoxynucleotides for the preparation of oligodeoxynucleotide-peptide hybrid molecules. Bioconjug Chem. 1998 Jul.-Aug.;9 (4):466-75.

Modifications of antisense molecules or other drugs that target the molecule to certain cells and enhance uptake of the oligonucleotide by said cells are described by Wang (1998).

The antisense oligonucleotides of the invention are generally provided in the form of pharmaceutical compositions. These compositions are for use by injection, topical administration, or oral uptake inter alia; see Example 7 for further information.

The mechanism of action of antisense RNA and the current state of the art on use of antisense tools is reviewed in Kumar et al (1998): Antisense RNA: function and fate of duplex RNA in cells of higher eukaryotes. Microbiol Mol Biol Rev. 1998 Dec.;62(4):1415-34. There are reviews on the chemical (Crooke, 1995: Progress in antisense therapeutics. Hematol Pathol. 1995;9(2):59-72; Uhlmann et al, 1990), cellular (Wagner, 1994: Gene inhibition using antisense oligodeoxynucleotides. Nature. 1994 Nov. 24;372(6504):333-5.) and therapeutic (Hanania, et al, 1995: Recent advances in the application of gene therapy to human disease. Am J Med. 1995 Nov.;99(5):537-52.; Scanlon, et al, 1995: Oligonucleotide-mediated modulation of mammalian gene expression. FASEB J. 1995 Oct.;9 (13):1288-96. ; Gewirtz, 1993: Oligodeoxynucleotide-based therapeutics for human leukemias. Stem Cells. 1993 Oct.;11 Suppl 3:96-103) aspects of this rapidly developing technology. The use of antisense oligonucleotides in inhibition of BMP receptor synthesis has been described by Yeh et al (1998): Inhibition of BMP receptor synthesis by antisense oligonucleotides attenuates OP-1 action in primary cultures of fetal rat calvaria cells. J Bone Miner Res. 1998 Dec.;13(12):1870-9.The use of antisense oligonucleotides for inhibiting the synthesis of the voltage-dependent potassium channel gene Kv1.4 has been described by Meiri et al (1998) Memory and long-term potentiation (LTP) dissociated: normal spatial memory despite CA1 LTP elimination with Kv1.4 antisense. Proc Natl Acad Sci U S A. 1998 Dec. 8;95(25):15037-42.The use of antisense oligonucleotides for inhibition of the synthesis of Bcl-x has been described by Kondo et al (1998): Antisense telomerase treatment: induction of two distinct pathways, apoptosis and differentiation. FASEB J. 1998 Jul.;12(10):801-11. The therapeutic use of antisense drugs is discussed by Stix (1998): Shutting down a gene. Antisense drug wins approval. Sci Am. 1998 Nov.;279(5):46, 50; Flanagan (1998) Antisense comes of age. Cancer Metastasis Rev. 1998 Jun.;17(2):169-76; Guinot et al (1998) Antisense oligonucleotides: a new therapeutic approach Pathol Biol (Paris). 1998 May;46(5):347-54, and references therein. Within a relatively short time, ample information has accumulated about the in vitro use of AS nucleotide sequences in cultured primary cells and cell lines as well as for in vivo administration of such nucleotide sequences for suppressing specific processes and changing body functions in a transient manner. Further, enough experience is now available from in vitro and in vivo in animal models and human clinical trials to predict human efficacy.

Novel BMP2A antisense fragments which may be used as the active ingredient in the pharmaceutical compositions and methods of treatment of the present invention are detailed in Table 2. In one embodiment, the antisense fragment of ID No 1 (SEQ ID NO: 46) is preferred; this antisense fragment was originally used to identify gene BMP2A as being involved in neurotoxic-induced apoptosis.

TABLE 2
BMP2A antisense oligonucleotides

| ID | SEQ | |
|---|---|---|
| No | ID | Sequence |
| 1 | 46 | 5′ GTTGTTTTCCCACTCGATTCTGGTAGTTCTTCCAAAGATTC |
| | | TTCATGGTGGAAGCTGCGCACAGTGTTGGCTAGGCTGGCTGCCC |
| | | TCTCCAACCC 3′ |
| 2 | 47 | 5′ AGCTTTTCCAAAAAGAACTATCAGGACATGGTTTCTCTTGA |
| | | AAACCATGTCCTGATAGTTCGGG 3′ |
| 3 | 48 | 5′ AGCTTTTCCAAAAATGACGAGAATGAAAAGGTTTCTCTTGA |
| | | AAACCTTTTCATTCTCGTCAGGG 3′ |
| 4 | 49 | 5′ AGCTTTTCCAAAAAGCTGTACCTTGACGAGAATTCTCTTGA |
| | | AATTCTCGTCAAGGTACAGCGGG 3′ |
| 5 | 50 | 5′ AGCTTTTCGAAAAACGACAGAACTCAGTGCTATTCTCTTGA |
| | | AATAGCACTGAGTTCTGTCGGGG 3′ |
| 6 | 51 | 5′ AGCTTTTCCAAAAAGGTCAACTCTGTTAACTCTTCTCTTGA |
| | | AAGAGTTAACAGAGTTGACCGGG 3′ |
| 7 | 52 | 5′ AGCTTTTCCAAAAACACTAATCATGCCATTGTTTCTCTTGA |
| | | AAACAATGGCATGATTAGTGGGG 3′ |
| 8 | 53 | 5′ AGCTTTTCCAAAAAGGGGTGGAATGACTGGATTTCTCTTGA |
| | | AAATCCAGTCATTCCACCCCGGG 3′ |
| 9 | 54 | 5′ AGCTTTTCCAAAAACCACAAAAGAGAAAAACGTTCTCTTGA |
| | | AACGTTTTTCTCTTTTGTGGGGG 3′ |
| 10 | 55 | 5′ AGCTTTTCCAAAAAGCATCCTCTCCACAAAAGATCTCTTGA |
| | | ATCTTTTGTGGAGAGGATGCGGG 3′ |
| 11 | 56 | 5′ AGCTTTTCCAAAAACTTCCACCATGAAGAATCTTCTCTTGA |
| | | AAGATTCTTCATGGTGGAAGGGG 3′ |
| 12 | 57 | 5′ AGCTTTTCCAAAAAGCAGCTTCCACCATGAAGATCTCTTGA |
| | | ATCTTCATGGTGGAAGCTGCGGG 3′ |
| 13 | 58 | 5′ AGCTTTTCCAAAAACTACATGCTAGACCTGTATTCTCTTGA |
| | | AATACAGGTCTAGCATGTAGGGG 3′ |
| 14 | 59 | 5′ AGCTTTTCCAAAAAGCATGTTCGGCCTGAAACATCTCTTGA |
| | | ATGTTTCAGGCCGAACATGCGGG 3′ |
| 15 | 60 | 5′ AGCTTTTCCAAAAAGTACCTTGACGAGAATGAATCTCTTGA |
| | | ATTCATTCTCGTCAAGGTACGGG 3′ |
| 16 | 61 | 5′ AGCTTTTCCAAAAACGATGCTGTACCTTGACGATCTCTTGA |
| | | ATCGTCAAGGTACAGCATCGGGG 3′ |
| 17 | 62 | 5′ AGCTTTTCCAAAAACTATCTCGATGCTGTACCTTCTCTTGA |
| | | AAGGTACAGCATCGAGATAGGGG 3′ |
| 18 | 63 | 5′ AGCTTTTCCAAAAATCAGTGCTATCTCGATGCTTCTCTTGA |
| | | AAGCATCGAGATAGCACTGAGGG 3′ |
| 19 | 64 | 5′ AGCTTTTCCAAAAAGAACTCAGTGCTATCTCGATCTCTTGA |
| | | ATCGAGATAGCACTGAGTTCGGG 3′ |
| 20 | 65 | 5′ AGCTTTTCCAAAAAAGAACTCAGTGCTATCTCGTCTCTTGA |
| | | ACGAGATAGCACTGAGTTCTGGG 3′ |
| 21 | 66 | 5′ AGCTTTTCCAAAAACTCTAAGATTCCTAAGGCATCTCTTGA |
| | | ATGCCTTAGGAATCTTAGAGGGG 3′ |

### Example 9

### Therapeutic Delivery of siRNA

Delivery systems aimed specifically at the enhanced and improved delivery of siRNA into mammalian cells have been developed. Shen et al (FEBS letters 539: 111-114 (2003)) described an adenovirus-based vector which efficiently delivers siRNAs into mammalian cells. Additional detail on viral-based siRNA delivery systems can be found in Xia et al., Nature Biotechnology 20: 1006-1010 (2002); and Reich et al., Molecular Vision 9: 210-216 (2003).

Sorensen et al. (J.Mol.Biol. 327: 761-766 (2003)) devised injection-based systems for systemic delivery of siRNAs to adult mice, by cationic liposome-based intravenous injection and/or intraperitoneal injection.

A system for efficient delivery of siRNA into mice by rapid tail vain injection has also been developed (Lewis et al., nature genetics 32: 107-108 (2002)).

Additionally, the peptide based gene delivery system MPG, previously used for DNA targeting, has been modified to be effective with siRNAs (Simeoni et al., Nuclaic Acids Research 31, 11: 2717-2724 (2003)).

Additional methods for delivery of siRNAs are described in Example 8, under the heading of delivery of AS fragments. Dosage and formulation of pharmaceuticals comprising siRNAs are discussed in Example 7.

### Example 10

### Experimental Models

CNS injury—The potential of the use of a BMP2A inhibitor for treating CNS injury is evaluated in animal models. The models represent varying levels of complexity, by comparison of control animals to the inhibitor treated animals. The efficacy of such treatment is evaluated in terms of clinical outcome, neurological deficit, dose-response and therapeutic window. Test animals are treated with a BMP2A inhibitor intravenously or subcutanously or per os. Control animals are treated with buffer or pharmaceutical vehicle only. Models used may be selected from the following:
1. Closed Head Injury (CHI)—Experimental TBI produces a series of events contributing to neurological and neurometabolic cascades, which are related to the degree and extent of behavioral deficits. CHI is induced under anesthesia, while a weight is allowed to free-fall from a prefixed height (Chen et al, J. Neurotrauma 13, 557, 1996) over the exposed skull covering the left hemisphere in the mideoronal plane.
2. Transient middle cerebral artery occlusion (MCAO)—a 90 to 120 minutes transient focal ischemia is performed in adult, male Sprague Dawley rats, 300-370 gr. The method employed is the intraluminal suture MCAO (Longa et al., Stroke, 30, 84, 1989, and Dogan et al., J. Neurochem. 72, 765, 1999). Briefly, under halothane anesthesia, a 3-0-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.
3. Permanent middle cerebral artery occlusion (MCAO)—occlusion is permanent, unilateral-induced induced by electrocoagulation of MCA. Both methods lead to focal brain ischemia of the ipsilateral side of the brain cortex leaving the contralateral side intact (control). The left MCA is exposed via a temporal craniectomy, as described for rats by Tamura A.et al., J Cereb Blood Flow Metab. 1981;1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

Evaluation Process The efficacy of the BMP2A inhibitor is determined by mortality rate, weight gain, infarct volume, short and long term clinical and neurophysichological and behavioral (including feeding behavior) outcomes in surviving animals. Infarct volumes are assessed histologically (Knight et al., Stroke, 25, 1252, 1994, and Mintorovitch et al., Magn. Reson. Med. 18, 39, 1991). The staircase test ( Montoya et al., J. Neurosci. Methods 36, 219, 1991) or the motor disability scale according to Bederson's method ( Bederson et al., Stroke, 17, 472, 1986) is employed to evaluate the functional outcome following MCAO. The animals are followed for different time points, the longest one being two months. At each time point (24 h, 1 week, 3, 6, 8 weeks), animals are sacrificed and cardiac perfusion with 4% formaldehyde in PBS is performed. Brains are removed and serial coronal 200 μm sections are prepared for processing and paraffin embedding. The sections are stained with suitable dyes such as TCC. The infarct area is measured in these sections using a computerized image analyzer.

Utilization of the BMP2A inhibitor treatment as exemplified in the above animal models provides new possibilities for treatment of human brain injury, whether acute or chronic.

### Example 11

### Screening Systems

The BMP2 gene or polypeptide may be used in a screening assay for identifying and isolating compounds which modulate its activity and, in particular, compounds which modulate neurotoxic stress or neurodegenerative diseases. The compounds to be screened comprise inter alia substances such as small chemical molecules, antibodies, antisense oligonucleotides, antisense DNA or RNA molecules, polypeptides and dominant negatives, and expression vectors.

Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. If the candidate polypeptide is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, and such cell-based systems are particularly relevant in order to directly measure the activity of candidate genes which are anti-apoptotic functional genes, i.e., expression of the gene prevents apoptosis or otherwise prevents cell death in target cells. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285,1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J.Neuroscience 18, 5285 and Tang et al (1997) J Clin. Invest. 100, 1180 for measurement of kinase activity. Although this is not relevant in the case of BMP2 which does not have a known enzymatic activity, there is a possibility that BMP2 interacts with an enzyme and regulates its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) or other species which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of apoptosis is also considered to be part of the present invention.

### Example 12

### Gene Therapy

The term “gene therapy” as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, antisense) the production of which in vivo is desired. For example, the genetic material of interest can encode a hormone, receptor, enzyme, polypeptide or peptide of therapeutic value. Alternatively, the genetic material of interest may encode a suicide gene. For a review see, in general, the text “Gene Therapy” (Advances in Pharmacology 40, Academic Press, 1997).

Gene therapy of the present invention can be carried out in vivo or ex vivo. Ex vivo gene therapy requires the isolation and purification of patient cells, the introduction of a therapeutic gene and the introduction of the genetically altered cells back into the patient. A replication-deficient virus such as a modified retrovirus can be used to introduce the therapeutic BMP2A cDNA or BMP2A antisense fragment into such cells. For example, mouse Moloney leukemia virus (MMLV) is a well-known vector in clinical gene therapy trials. See, e.g., Boris-Lauerie et al., Curr. Opin. Genet Dev., 3, 102-109 (1993).

In contrast, in vivo gene therapy does not require isolation and purification of a patient's cells. The therapeutic gene or fragment such as an antisense fragment is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. Another approach is administration of “naked DNA” in which the therapeutic gene or fragment such as an antisense fragment is directly injected into the bloodstream or muscle tissue. Still another approach is administration of “naked DNA” in which the therapeutic gene or fragment such as an antisense fragment is introduced into the target tissue by microparticle bombardment using gold particles coated with the DNA.

Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Cell types useful for gene therapy of the present invention include lymphocytes, hepatocytes, myoblasts, fibroblasts, and any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846. For a review of the subject of gene therapy, in general, see the text “Gene Therapy” (Advances in Pharmacology 40, Academic Press, 1997).

1-25. (canceled)

26. A double stranded oligoribonucleotide, wherein one strand comprises consecutive nucleotides having, from 5′ to 3′, the sequence set forth in any of SEQ ID NOS: 25-45 or SEQ ID NO: 67 or a homolog thereof and wherein a base is altered in up to 2 of the nucleotides in each terminal region.

27. A vector comprising an oligoribonucleotide of claim 26.

28. A composition comprising an oligoribonucleotide of claim 26.

29. A composition comprising a vector of claim 27.

30. A method of treating a neurodegenerative disease in a subject which comprises administering to the subject a therapeutically effective amount of a pharmaceutical composition comprising a BMP2A inhibitor so as to thereby treat the subject.

31. The method of claim 30, wherein the pharmaceutical composition comprises an oligoribonucleotide which down regulates the expression of the BMP2A gene by at least 50% as compared to a control.

32. The method of claim 30, wherein the BMP2A inhibitor is an antisense oligonucleotide.

33. The method of claim 30, wherein the BMP2A inhibitor is a BMP2A siRNA.

34. The method of claim 33, wherein the siRNA is a double stranded oligoribonucleotide wherein one strand comprises consecutive nucleotides having, from 5′ to 3′, the sequence set forth in any of SEQ ID NOS: 25-45 or SEQ ID NO: 67 or a homolog thereof and wherein a base is altered in up to 2 of the nucleotides in each terminal region.

35. The method of claim 33, wherein the siRNA has a sequence selected from the group SEQ ID NOS: 1-2, 4-6, 14-16, and 18-22 which are set forth in Table 1.

36. The method of claim 30, wherein the disease is a stroke.

37. An oligonucleotide comprising consecutive nucleotides, the sequence of which is set forth in any of SEQ ID NOS: 3-45 or SEQ ID NO: 67.

38. A vector comprising an oligonucleotide of claim 37.

39. A pharmaceutical composition comprising an oligonucleotide of claim 37 and a pharmaceutically acceptable carrier.

40. A pharmaceutical composition comprising a vector of claim 37 and a pharmaceutically acceptable carrier.

41. The method of claim 31, wherein the disease is a stroke.

42. The method of claim 32, wherein the disease is a stroke.

43. The method of claim 33, wherein the disease is a stroke.

44. The method of claim 34, wherein the disease is a stroke.

45. The method of claim 35, wherein the disease is a stroke.
